(19) United States
(12) Patent Application Publication
(10) Pub. No.: US 2015/0105460 A1
(43) Pub. Date: Apr. 16, 2015

(54) LABDANE DITERPENOID COMPOUNDS, SEMEN BIOTAE EXTRACT, AND PREPARATION METHOD AND USE THEREOF

(71) Applicants: Weiwei Su, Guangzhou (CN); Yonggang Wang, Guangzhou (CN); Fengyin Liang, Guangzhou (CN); Ning Wang, Guangzhou (CN); Zhong Pei, Guangzhou (CN); Haibin Liu, Guangzhou (CN)

(72) Inventors: Weiwei Su, Guangzhou (CN); Yonggang Wang, Guangzhou (CN); Fengyin Liang, Guangzhou (CN); Ning Wang, Guangzhou (CN); Zhong Pei, Guangzhou (CN); Haibin Liu, Guangzhou (CN)

(21) Appl. No.: 14/404,644

(22) PCT Filed: Jun. 4, 2012
(86) PCT No.: PCT/CN2012/076443

§ 371 Date: Dec. 1, 2014

(51) Int. Cl. A61K 36/14; C07C 62/36; C07C 62/32; C07C 61/35; C07D 307/58; C07C 62/30; C07C 49/755; C07D 307/60; C07C 62/38

(52) U.S. Cl. CPC A61K 36/14; C07D 307/60; C07C 62/36; C07C 62/32; C07C 62/38; C07D 307/58; C07C 62/30; C07C 49/755; C07C 61/35
USPC 514/473; 549/318; 562/501; 514/557; 549/479; 514/574; 568/373; 514/691; 562/499; 549/478; 568/817; 514/729; 549/499; 514/461

(57) ABSTRACT

Provided in the present invention are various labdane diterpenoid compounds and derivatives thereof for use in preparation of a pharmaceutical composition for preventing or treating neurodegenerative diseases. Also provided in the present invention is a semen biotae extract comprising one or more labdane diterpenoid compounds and derivatives thereof for use in preparation of a pharmaceutical composition for preventing or treating neurodegenerative diseases.

### FIELD OF THE INVENTION

The present invention relates to labdane diterpenoid compounds, semen biotae (Semen biotae) extracts as well as preparation methods and uses of the same.

### BACKGROUND OF THE INVENTION

Neuro degenerative diseases (Neuro degenerative diseases, NDD) are a type of chronic and progressive disease, and primarily include Alzheimer's disease (Alzheimer's disease, AD), Pakinson's disease (Pakinson's disease, PD), Huntington disease (Huntington disease, HD), Amyotrophic lateral sclerosis (Amyotrophic lateral sclerosis, ALS), and the like. This type of diseases has complex pathological and physiological process, and they have different lesion regions and causes. However, they are in common in degenerative lesions of nerve cells. Patients suffering from neuro degenerative diseases often suffer from memory deterioration, cognitive difficulties, dementia, motion balance disorder, loss of exercise ability and the like. Moreover, such patients suffer from serious decline of quality of life, long course of diseases, and impossibility of cure, which bring huge economic and psychological pressure to the patients and the family. Diseases relating to aging now become a major challenge to scientists with the global increasing aging degree.

By now, there is no measure to effectively control the progress of this type of disease in clinic. Currently, most of the drugs for treating neuro degenerative diseases only alleviate the symptoms rather than cure the diseases, and they have significant side effects. For example, by now there is no drug or treating measure which completely blocks the progressing of PD; tacrine (tacrine), drug for treating AD, has been eliminated from selection for its toxic and side effects to liver as well as significant drug-drug interactions; the side reactions of donepezil (donepezil), rivasrigmine (rivasrigmine) and galantamine (galantamine) are primarily nausea, vomiting, diarrhea and anorexia. Therefore, there is a pressing need in a drug for treating neuro degenerative diseases, which protects the nerve cells and changes the progress of the disease at the same time, and which has no side effects.

Semen biotae (Semen biotae), also named seed of cypress, seed of platycladusorientalis, and the like, is the mature seed of platycladusorientalis, cupressaceae family, and is primarily produced in Shandong, Henan, Hebei, Shaanxi, Hubei, Gansu and so on. Semen biotae is a frequently used Chinese traditional herb medicine. The use of semen biotae is often observed in Chinese traditional herb medicines made up of two or more ingredients, such as BaiziYangxin pills (semen biotae, codonopsis, cinnamon, polygala, schisandra, semen ziziphispinosae, cinnabar, astragalus, rhizomachuanxiong, angelica, fermented pinellia, poria, and licorice), which is used in patients lack of heart-qi and patients with insomnia or amnesia. A healthy drink for improving memory and preventing Alzheimer's disease include 21 herb medicines like semen biotae, fructusrubi, poria, polygala and so on. There are few reports on the use of semen biotae extract in treating neuro degenerative diseases.

### SUMMARY OF THE INVENTION

One aim of the present invention is to provide a pharmaceutical composition, which is capable of preventing or treating neuro degenerative diseases.

The present invention provides labdane diterpenoid compounds, which includes one or more ingredients of labdane diterpenoids shown by the following two formulas:

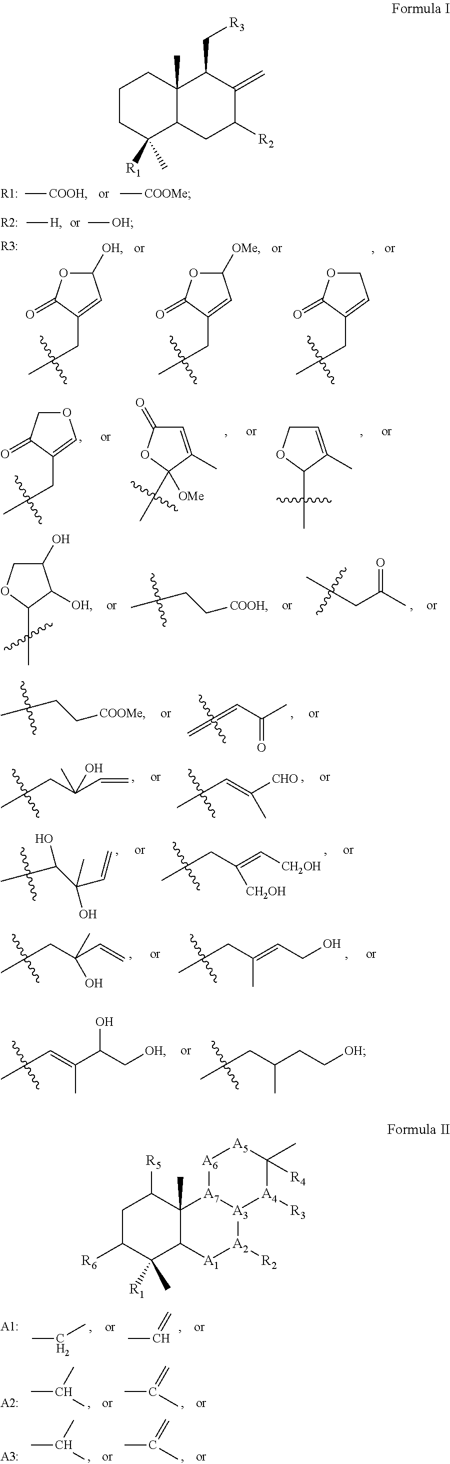

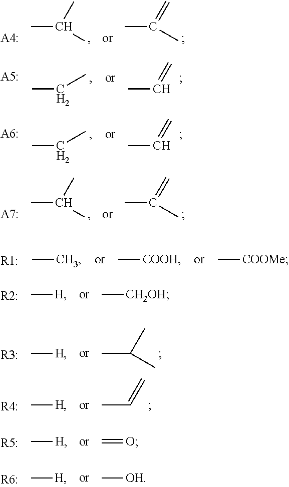

The labdane diterpenoids include:

15-Hydroxypinusolidic acid;

15-Methoxyl abietic acid;

1-Naphthalenecarboxylic acid, decahydro-1,4a-dimethyl-6-methylene-5-(3-oxobutyl)-, (1S, 4aR, 5S, 8aR)-;

12, 13-Dihydroxylabda-8(17)-14-dien-19-oic acid;

pinusolidic acid;

7β, 13 S-Dihydroxylabda-8(17)-14-dien-19-oic acid;

1-Naphthalenecarboxylic acid, decahydro-1,4a-dimethyl-6-methylene-5-[(1E)-3-oxo-1-butenyl]-, (1S, 4aS, 5S, 8aR)-;

Isopinusolide;

Platyclolactonic acid;

14,15-bisnor-8(17)-labdene-16,19-dioic acid;

1-oxo-3β-hydroxytotarol;

6,7-dehydro-sandaracopimaric acid;

Sandaracopimaric acid;

12,13,14-trihydroxyl-12,15-epoxyabda-8(17)-en-19 oic acid;

Pinusolide;

Platyclolactonic acid methyl ester;

Isopimaric acid;

7α-Hydroxysandaracopimaric acid;

15,16-Dihydroxy-8(17), 13(E)-labdadien-19-oic acid;

13-Epicupressic acid;

Isocupressic acid;

Sandaracopimaradiene-3β,18-diol;

14(R), 15-Dihydroxy-8(17), 12(E)-labdadien-19-oic acid;

14,15-Bisnor-8(17), 12E-labdadien-19-oic acid methyl ester;

Naphthalenecarboxylic acid, decahydro-1,4a-dimethyl-6-methylene-5-[(2E)-3-methyl-4-oxo-2-butenyl]-, (1S, 4aR, 5S, 8aR)-;

16-Methyl-12,15-epoxy-8(17), 13-labdadien-19-oic acid;

Imbricatolic acid;

and the like or derivatives of the labdane diterpenoid compounds above;

wherein the labdane diterpenoid compounds or derivatives thereof is used to prevent or treat neuro degenerative diseases.

The present invention also provides labdane diterpenoid compounds or derivatives thereof which are obtained by separating from semen biotae extracts, or obtained by derivation of precursor, semi-synthesis or total synthesis.

The present invention also provides semen biotae extracts, and said semen biotae extracts comprise one or more labdane diterpenoid compounds or derivatives thereof described above.

The present invention also provides the preparation process of semen biotae extracts:

providing powder of semen biotae;

extracting with carbon dioxide under supercritical states to remove semen biotae oil, thereby obtaining herb residue;

adding ethanol to said herb residue, and extracting the same to obtain an extractum with a relative density over 1.20;

solving said extratumin in water;

extracting with ethyl acetate to obtain the semen biotae extract. The single semen biotae diterpenoid compounds are separated by column chromatography methods.

The present invention also provides a pharmaceutical composition for use in preventing or treating neuro degenerative diseases, wherein said pharmaceutical composition comprises one or more labdane diterpenoid compounds or derivatives thereof or semen biotae extracts described above.

The present invention is further illustrated in connection with the accompanying figures and specific embodiments. However, the present invention is not limited within these embodiments. Any improvement or alteration made on the basis of the principle of the present invention also belongs to the scope of the claims of the present invention.

### BRIEF DESCRIPTION OF THE DRAWING

FIG. 1 shows the chemical formula of 27 labdane diterpenoid compounds; the names of each labdane diterpenoid compound are shown below:

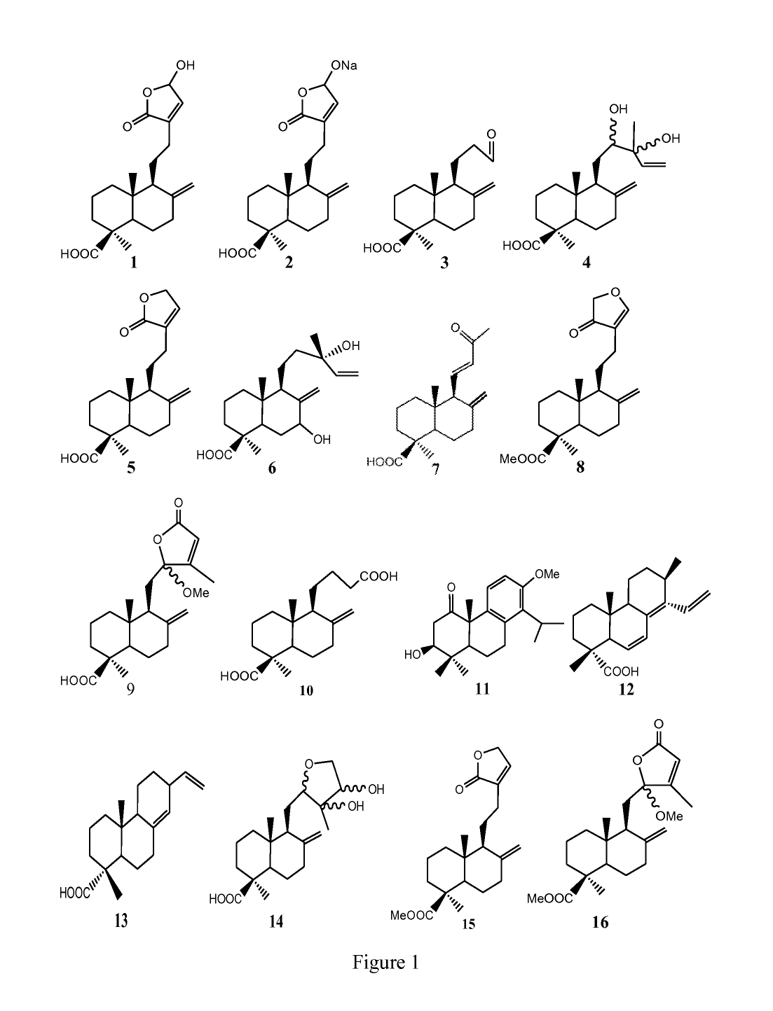

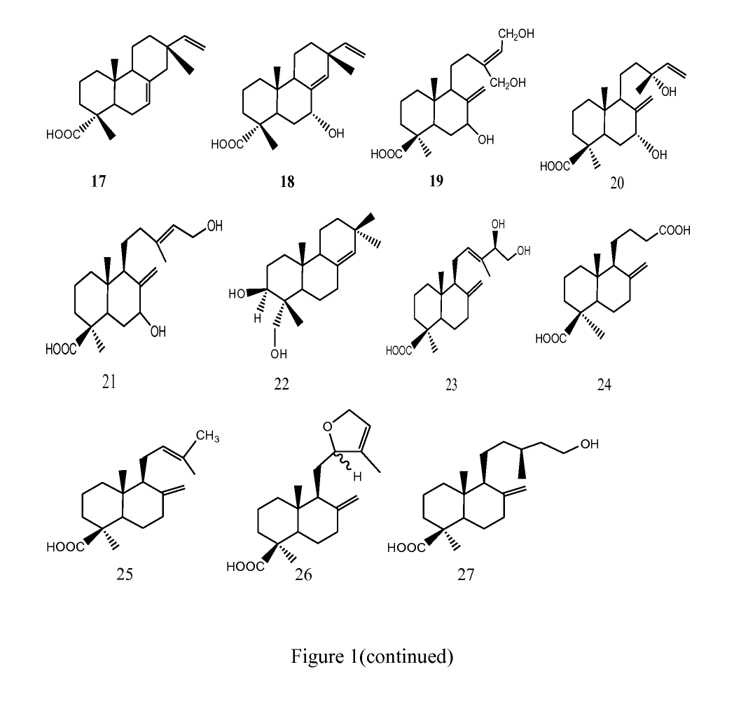

| Serial | Name |
|---|---|
| 1 | 15-Hydroxypinusolidic acid |
| 2 | 15-Methoxypinusolidic acid |
| 3 | 1-Naphthalenecarboxylic acid, decahydro -1,4a-dimethyl-6- |
| | methylene-5-(3-oxobutyl)-, (1S,4aR,5S,8aR)- |
| 4 | 12,13-Dihydroxylabda-8(17),14-dien-19-oic acid |
| 5 | Pinusolidic acid |
| 6 | 7β,13S-dihydroxylabda-8(17),14-dien-19-oic acid |
| 7 | 1-Naphthalenecarboxylic acid, decahydro-1,4a-dimethyl-6- |
| | methylene-5-[(1E)-3-oxo-1-butenyl]-, (1S,4aS,5S,8aR)- |
| 8 | Isopinusolide |
| 9 | Platyclolactonic Acid |
| 10 | 14,15-bisnor-8(17)-labdene-16,19-dioic acid |
| 11 | 1-oxo-3β-hydroxytotarol |
| 12 | 6,7-Dehydrosandarapimaric Acid |
| 13 | Sandaracopimaric acid |
| 14 | 12,13,14-trihydroxylabda-12,15-epoxy-8(17)-en-19-oic acid |
| 15 | Pinusolide |
| 16 | Platyclolactonic Acid Methyl Ester |
| 17 | Isopimaric acid |
| 18 | 7α-Hydroxysandaracopimaric acid |
| 19 | 15,16-Dihydroxy-8(17),13(E)-labdadien-19-oic acid |
| 20 | 13-Epicupressic acid |
| 21 | Isocupressic acid |
| 22 | Sandaracopimaradiene-3β,18-diol |
| 23 | 14(R),15-Dihydroxy-8(17),12(E)-labdadien-19-oic acid |
| 24 | 14,15-Bisnor-8(17),12E-labdadien-19-oic acid methyl ester |
| 25 | Naphthalenecarboxylic acid, decahydro-1,4a-dimethyl-6-meth- |
| | ylene-5-[(2E)-3-methyl-4-oxo-2-butenyl]-, (1S,4aR,5S,8aR)- |
| 26 | 16-Methyl-12,15-epoxy-8(17),13-labdadien-19-oic acid |
| 27 | Imbricatolic acid |

### DETAILED DESCRIPTION OF THE INVENTION

The present invention gains significant advances in developing drugs for preventing or treating neuro degenerative diseases. Different experiments prove that semen biotae extracts and labdane diterpenoid compounds may significantly increase the activity of PD model cells, delay the paralysis time of AD model nematode, elongate the life of nematode, and delay the aging of neuroprotective activities.

### EXAMPLE 1

### Preparation of Semen Biotae Extracts

Obtaining semen biotae and mashing the sample. Extracting the sample with carbon dioxide under supercritical states to remove semen biotae oil. The herb residue is extracted with 95% ethanol, and each time 10 equivalent volume 95% ethanol is added. The mixture is heated to reflux, and then recover solvent from the mixture to obtain extractum with a relative density over 1.20. The extractum is solved in water, and the obtained mixture is extracted with ethyl acetate for three times. Then recover solvent from the extract liquor to obtain semen biotae diterpene extracts. Semen biotae diterpenoid compounds are obtained by separating with column chromatography.

### EXAMPLE 2

### Anti-PD Model Cell Experiments of Semen Biotae Extracts and Diterpene Compounds

The semen biotae extracts and semen biotae diterpenoid compounds in Example 1 are used in the experiments.

PC12 cells (rat adrenal pheochromocytoma cell strain), which show many similar characters with dopaminergic neurons, is cultured in DMEM medium (which contains 5% horse serum, 5% fetal bovine serum, 100 U/mLpenicillin, 100 g/mL streptomycin), in an incubator under 37° C., saturated humidity, and 5% CO₂. The medium is replaced every 2-3 days, and the cells are subcultured when the cells cover 70-80% area of the plate. Cells in log phase and under fine growth conditions are chosen, and inoculated in 96 well plate at 1-5×10⁵and 100 μl per well. After culturing under 37° C. for 24 hours and the cells adhere to the walls of the wells, MPP medium is added to the model group, a medium containing MPP and semen biotae extracts/diterpenoid compounds is added to experimental group, and a medium with no drug is added to the blank control group. Then continue to culture the media in the incubator for 24 hours. MTT assay is used to evaluate cell activity.

The results are shown in Table 1, demonstrating that semen biotae extracts and diterpene compounds significantly enhance the cell activities in the PC12 cells injured by MPP⁺.

TABLE 1
Effects of biotae extracts and diterpene compounds
on the injuries of PC12 cells caused by MPP⁺

| | average OD | Relative Cell |
|---|---|---|
| Group | value | Activity % |
| Normal Control | 2.198 ± 0.046 | 100 |
| Model group | 1.061 ± 0.035 | 48.3 |
| Semen Biotae Extract | 1.543 ± 0.066** | 70.2 |
| 15-hydroxypinusolidic acid | 1.585 ± 0.057** | 72.1 |
| 1-Naphthalenecarboxylic acid, decahydro- | 1.391 ± 0.038* | 63.3 |
| 1,4a-dimethyl-6-methylene-5-(3-oxobutyl)-, | | |
| (1S,4aR,5S,8aR)- | | |
| 6,7-Dehydrosandarapimaric Acid | 1.435 ± 0.044** | 65.3 |
| 14,15-bisnor-8(17)-labdene-16,19-dioic acid | 1.292 ± 0.072* | 58.8 |
| 1-oxo-3β-Hydroxytotarol | 1.448 ± 0.057** | 65.9 |
| Pinusolidic acid | 1.224 ± 0.057* | 55.7 |
| 12,13,14-trihydroxylabda-12,15-epoxy-8(17)-en-19- | 1.404 ± 0.057** | 63.9 |
| oic acid | | |
| 7β,13S-dihydroxylabda-8(17),14-dien-19-oic acid | 1.359 ± 0.045* | 61.8 |
| Sandaracopimaradiene-3β,18-diol | 1.652 ± 0.015** | 75.1 |
| 14(R),15-Dihydroxy-8(17),12(E)-labdadien-19-oic | 1.346 ± 0.059* | 61.2 |
| acid | | |
| 14,15-Bisnoar-labdene-8(17),12E-dien19-oic Acid | 1.536 ± 0.035** | 69.9 |
| methyl Ester | | |
| Naphthalenecarboxylic acid, decahydro-1, | 1.236 ± 0.085* | 56.2 |
| 4a-dimethyl-6-methylene-5-[(2E)-3-methyl- | | |
| 4-oxo-2-butenyl]-, (1S,4aR,5S,8aR)- | | |

*means that P < 0.05 comparing with the model group;
**means P < 0.01 comparing with the model group.

### EXAMPLE 3

### Anti-PD Model Elegan Experiments of Semen Biotae Extracts and Diterpenoid Compounds

The semen biotae extracts and semen biotae diterpenoid compounds in Example 1 are used in the experiments.

Caenorhaditiselegans (Caenorhaditiselegans) transgene strain CL4176 (expressing Aβ protein under temperature inducing) are synchronized and cultured under 15° C. to L1 phase, and then added into coated OP50 microbial medium dishes containing the extracted drugs with different concentrations, at around 25/dish. Three dishes are used in one group. The dishes are cultured under 15° C. for 12 hours and then cultures under 26° C. 36 hours after the temperature is changed, the paralyses of the elegans are observed. Count every three hours until all the elegans are paralyzed. The results are shown in the table below, demonstrating that semen biotae extracts and diterpenoid compounds both significantly delay the paralysis of CL 4176 elegan caused by Aβ.

TABLE 2
Effect of semen biotae extracts and diterpenoid compounds
on the paralysis of AD model elegans.

| | Average Living |
|---|---|
| Group | Time (h) |
| Blank Control | 49.503 ± 0.445 |
| Semen biotae extracts | 54.823 ± 0.377** |
| pinusolide | 54.003 ± 0.287** |
| Platyclolactonic Acid Methyl Ester | 51.393 ± 0.487* |
| Isopimaric acid | 52.785 ± 0.432* |
| 7α-Hydroxysandaracopimaric acid | 53.067 ± 0.455** |
| 15,16-Dihydroxy-8(17),13(E)-labdadien-19-oic acid | 51.217 ± 0.397* |
| 13-Epicupressic acid | 54.015 ± 0.432** |
| Isocupressic acid | 53.879 ± 0.414** |
| 15-Methoxypinusolidic acid | 55.012 ± 0.399** |
| Sandaracopimaric acid | 53.616 ± 0.443** |
| Isopinusolide | 53.988 ± 0.320** |
| 12,13-Dihydroxylabda-8(17),14-dien-19-oicacid | 51.008 ± 0.434* |
| 15-Hydroxypinusolidic acid | 51.575 ± 0.257* |
| 1-Naphthalenecarboxylic acid, decahydro-1, | 50.991 ± 0.568* |
| 4a-dimethyl-6-methylene-5-(3-oxobutyl)-, | |
| (1S,4aR,5S,8aR)- | |
| 6,7-Dehydrosandarapimaric Acid | 52.435 ± 0.484** |
| 14,15-bisnor-8(17)-labdene-16,19-dioic acid | 51.892 ± 0.322* |
| 1-oxo-3β-hydroxytotarol | 53.478 ± 0.357** |
| 2-butenyl]-1-naphthoic acid | 52.136 ± 0.415* |
| 16-Methyl-12,15-epoxy-8(17),13-labdadien-19-oic | 54.336 ± 0.377** |
| acid | |
| Imbricatolic acid | 55.857 ± 0.428** |

*Pmeans that P < 0.05 comparing with the model group;
**means P < 0.01 comparing with the model group.

### EXAMPLE 4

### Anti-Elegan Aging Model Experiments of Semen Biotae Extracts and Diterpenoid Compounds

The semen biotae extracts and diterpenoid compounds in Example 1 are used in the experiments.

N2 elegans are synchronized to L4 phase, and then OP50 containing biotae extracts and diterpenoid compounds with different concentrations are coated onto NGM plates. Three plates are employed in one experimental group, and each plate includes about 25 elegans. The number of live elegans is recorded every day, and the elegans are transferred into a new NGM plate when the food is used up or there is mold contamination. The experiment pends till all the elegans are dead. The results are shown in the table below, demonstrating that semen biotae extracts and diterpenoid compounds significantly increase the average lifetime of elegans.

TABLE 3
Anti-Elegan Aging Experiments of Semen Biotae
Extracts and Diterpenoid Compounds

| | Average Lifetime |
|---|---|
| Group | (d) |
| Blank Control | 13.786 ± 0.512 |
| Semen Biotae Extracts | 17.219 ± 0.355** |
| Sandaracopimaradiene-3β,18-diol | 14.652 ± 0.355* |
| 14(R),15-Dihydroxy-8(17),12(E)-labdadien-19-oic | 16.046 ± 0.459* |
| acid | |
| 14,15-Bisnor-8(17),12E-labdadien-19-oic acid | 15.536 ± 0.435* |
| methyl ester | |
| Naphthalenecarboxylic acid, decahydro-1, | 16.136 ± 0.375** |
| 4a-dimethyl-6-methylene-5-[(2E)-3-methyl- | |
| 4-oxo-2-butenyl]-, (1S,4aR,5S,8aR)- | |
| 16-methyl-12,15-epoxyabda-8(17)-13-dien-19-oic | 16.336 ± 0.370** |
| acid | |
| Imbricatolic acid | 15.857 ± 0.428* |
| Platyclolactonic Acid | 16.432 ± 0.501** |
| 1-Naphthalenecarboxylic acid, decahydro-1, | 15.896 ± 0.375* |
| 4a-dimethyl-6-methylene-5-[(1E)-3-oxo-1-butenyl]-, | |
| (1S,4aS,5S,8aR)- | |
| pinusolide | 17.003 ± 0.487** |
| Platyclolactonic Acid Methyl Ester | 16.384 ± 0.387** |
| Isopimaric acid | 16.765 ± 0.442** |
| 7α-Hydroxysandaracopimaric acid | 16.076 ± 0.457* |
| 15,16-Dihydroxy-8(17),13(E)-labdadien-19-oic acid | 15.717 ± 0.396* |
| 13-Epicupressic acid | 15.045 ± 0.352** |
| Isocupressic acid | 14.889 ± 0.414* |
| 15-Methoxypinusolidic acid | 17.018 ± 0.389** |
| Sandaracopimaric acid | 16.716 ± 0.449** |
| Isopinusolide | 15.928 ± 0.370* |
| 12,13-Dihydroxylabda-8(17),14-dien-19-oic acid | 14.608 ± 0.414* |

*means that P < 0.05 comparing with the model group;
**means P < 0.01 comparing with the model group.

What is claimed is:

1. A labdane diterpenoid compound of Formula I or Formula II: 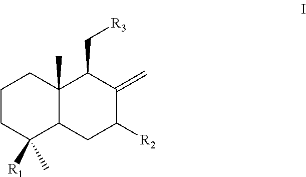
wherein: 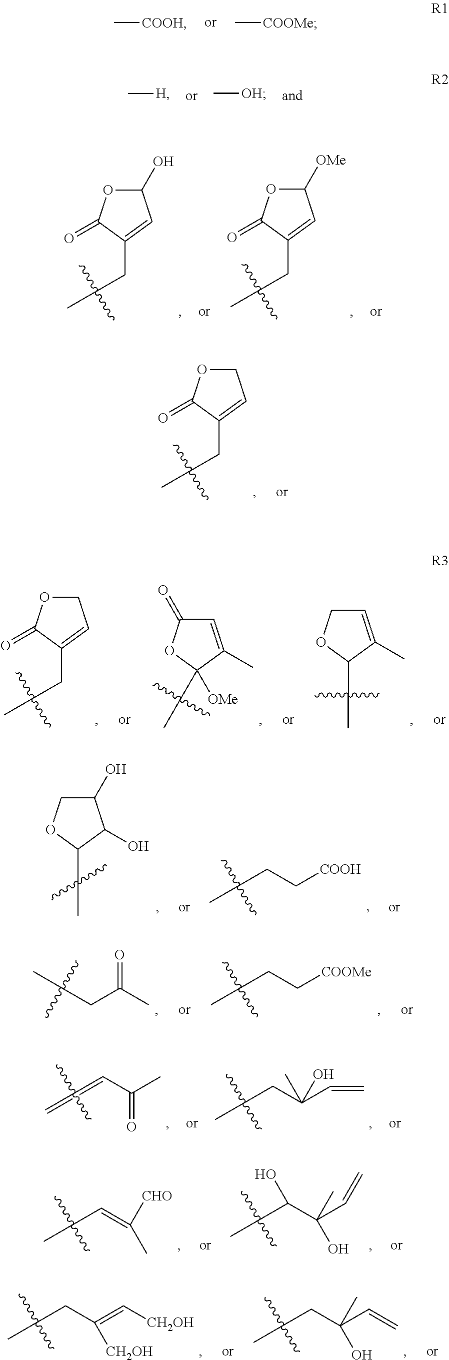 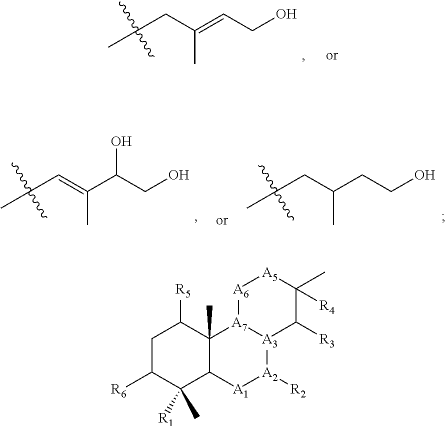
wherein: 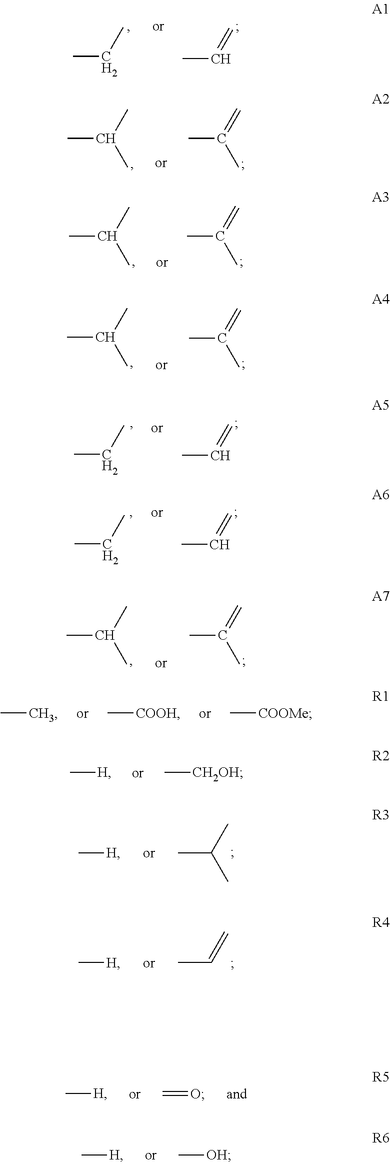
wherein the above labdane diterpenoid compound or derivatives thereof are used to prevent or treat neuro degenerative diseases.

2. The labdane diterpenoid compound according to claim 1, comprising
15-Hydroxypinusolidic acid;
15-Methoxyl abietic acid;
1-Naphthalenecarboxylic acid, decahydro-1,4a-dimethyl-6-methylene-5-(3-oxobutyl)-, (1S, 4aR, 5S, 8aR)-;
12,13-Dihydroxylabda-8(17)-14-dien-19-oic acid;
pinusolidic acid;
7β,13S-Dihydroxylabda-8(17)-14-dien-19-oic acid;
1-Naphthalenecarboxylic acid, decahydro-1,4a-dimethyl-6-methylene-5-[(1E)-3-oxo-1-butenyl]-, (1S, 4aS, 5S, 8aR)-;
Isopinusolide;
Platyclolactonic acid;
14,15-bisnor-8(17)-labdene-16,19-dioic acid;
1-oxo-3β-hydroxytotarol;
6,7-dehydro-sandaracopimaric acid;
Sandaracopimaric acid;
12,13,14-trihydroxyl-12,15-epoxyabda-8(17)-en-19-oic acid;
Pinusolide;
Platyclolactonic acid methyl ester;
Isopimaric acid;
7α-Hydroxysandaracopimaric acid;
15,16-Dihydroxy-8(17),13(E)-labdadien-19-oic acid;
13-Epicupressic acid;
Isocupressic acid;
Sandaracopimaradiene-3β,18-diol;
14(R),15-Dihydroxy-8(17),12(E)-labdadien-19-oic acid;
14,15-Bisnor-8(17),12E-labdadien-19-oic acid methyl ester;
Naphthalenecarboxylic acid, decahydro-1,4a-dimethyl-6-methylene-5-[(2E)-3-methyl-4-oxo-2-butenyl]-, (1S, 4aR, 5S, 8aR)-;
16-Methyl-12,15-epoxy-8(17),13-labdadien-19-oic acid;
Imbricatolic acid; and
the like or derivatives of the labdane diterpenoid compounds above.

3. The labdane diterpenoid compound according to claim 1, wherein the labdane diterpenoid compound and derivatives thereof are obtained by separating from semen biotae extracts, or obtained by derivation of precursor, semi-synthesis or total synthesis.

4. The labdane diterpenoid compound according to claim 3, wherein the semen biotae extracts are prepared by a process comprising:
providing powder of semen biotae;
extracting with carbon dioxide under supercritical states to remove semen biotae oil, thereby obtaining herb residue;
adding ethanol to said herb residue, and extracting the same to obtain an extractum with a relative density over 1.20;
solving said extratumin in water;
extracting with ethyl acetate to obtain the semen biotae extract; and
separating the single semen biotae diterpenoid compounds are ed by column chromatography methods.

5. A semen biotae extract, comprising one or more labdane diterpenoid compounds of claim 1 or derivatives thereof.

6. The semen biotae extract according to claim 5, wherein the semen biotae extract is prepared by a process comprising:
providing powder of semen biotae;
extracting with carbon dioxide under supercritical states to remove semen biotae oil, thereby obtaining herb residue;
adding ethanol to said herb residue, and extracting the same to obtain an extractum with a relative density over 1.20;
solving said extratum in water;
extracting with ethyl acetate to obtain the semen biotae extract; and
separating the single semen biotae diterpenoid compounds by column chromatography methods.

7. A pharmaceutical composition for use in preventing or treating neuro degenerative diseases, wherein the pharmaceutical composition comprises one or more labdane diterpenoid compounds of claim 1 or derivatives thereof.

8. The pharmaceutical composition according to claim 7, wherein the one or more labdane diterpenoid compounds or derivatives thereof are derived from semen biotae extracts.

9. The pharmaceutical composition according to claim 7, wherein the pharmaceutical composition is in the form of pills, powder, tablets, capsules, granules, or injection.
